(19) United States
(12) Patent Application Publication
(10) Pub. No.: US 2007/0180047 A1
(43) Pub. Date: Aug. 2, 2007

(54) System and method for providing authentication of remotely collected external sensor measures

(76) Inventors: Yanting Dong, Shoreview, MN (US); Todd P. Carpenter, St. Paul, MN (US); Quan Ni, Shoreview, MN (US); Kenneth P. Hoyme, Plymouth, MN (US)

Correspondence Address: CASCADIA INTELLECTUAL PROPERTY, 500 UNION STREET, STE.1005, SEATTLE, WA, 98101, US

(21) Appl. No.: 11/301,214

(22) Filed: Dec. 12, 2005

(51) Int. Cl. G06F 15/16; G06F 19/00; A61B 5/00

(52) U.S. Cl. 709/217; 128/903; 600/300; 705/3

(57) ABSTRACT

A system and method for providing authentication of remotely collected external sensor measures is presented. Physiological measures are collected from a source situated remotely from a repository for accumulating such collected physiological measures. An identification of the source from which the physiological measures were collected is determined against authentication data that uniquely identifies a specific patient. The physiological measures are forwarded to the repository upon authenticating the patient identification as originating from the specific patient.

### FIELD OF THE INVENTION

The present invention relates in general to external sensor authentication and, specifically, to a system and method for providing authentication of remotely collected external sensor measures.

### BACKGROUND OF THE INVENTION

Remote patient management has become increasingly attractive as an alternative to routine clinical follow-up in light of trending increases in healthcare costs. Remote patient management enables a clinician, such as a physician, nurse, or other healthcare provider, to follow patient well-being through homecare medical devices that can collect and forward patient data without requiring the presence or assistance of medical personnel. Advances in automation have encouraged such self-care solutions and public data communications networks, in particular, the Internet, have made ready data retrieval and patient communication viable and widely available.

To participate in remote patient management, each patient installs an at-home medical device, such as a patient management device, for collecting quantitative patient data measured by external sensors, such as a weight scale, blood pressure cuff, pulse oximeter, or glucometer, and for connecting to a centralized patient management facility, frequently implemented as a server accessible over the Internet. Other devices, such as a personal computer, can. measure and report qualitative patient data. In addition, implantable medical devices (IMDs), for example, pacemakers and implantable defibrillators, are beginning to include the capability to work with at-home medical devices.

To succeed, remote patient management must be user-friendly to encourage regular use. Difficulties in use will discourage patients and decrease the effectiveness of treatment and the benefit received. Ideally, remote patient management devices should introduce no more than minimal inconvenience, such as experienced when using a bathroom scale or thermometer, and will accommodate the needs of the infirm, elderly and physically challenged. Additionally, these devices should transparently manage spurious data, such as resulting from unauthorized use and from use by sources other than the patient, because raw patient data cannot easily be associated with a specific authorized patient. Conventional remote patient management devices assume that the patient is the only user and rely on implicit patient identification.

U.S. Pat. No. 6,168,563, to Brown, discloses a system and method that enables a healthcare provider to monitor and manage a health condition of a patient. A clearinghouse computer communicates with the patient through a data management unit, which interactively monitors the patient's health condition by asking questions and receiving answers that are supplied back to the clearinghouse computer. Patient information may also be supplied by physiological monitoring devices, such as a blood glucose monitor or peak-flow meter. Healthcare professionals can access the patient information through the clearinghouse computer, which can process, analyze, print, and display the data. However, Brown fails to disclose specific controls to ensure proper patient identification prior to accepting data from the data management unit.

U.S. Pat. No. 6,416,471, to Kumar et al. (“Kumar”), discloses a portable remote patient telemonitoring device. A disposable sensor band with electro-patches detects and transmits vital signs data to a signal transfer unit, which can be either be worn or positioned nearby the patient. The base station receives data transmissions from the signal transfer unit for transferring the collected data to a remote monitoring station. Indications are provided to a patient from a base station when threshold violations occur. However, Kumar fails to disclose authenticating the identity of the patient prior to receiving collected data from the base station.

U.S. Pat. No. 6,024,699, to Surwit et al. (“Surwit”), discloses a central data processing system configured to communicate with and receive data from patient monitoring systems, which may implement medical dosage algorithms to generate dosage recommendations. Blood from a pricked finger may be read on a chemically treated strip for review at the central data processing system. Modifications to medicine dosages, the medicine dosage algorithms, patient fixed or contingent self-monitoring schedules, and other treatment information are communicated. However, Surwit fails to disclose identifying the patient submitting the sample through each patient monitoring system.

Therefore, there is a need for providing an automated determination of patient identification associated with patient data collected by remote external and unsupervised sensors to ensure the integrity of the data received. Preferably, such an approach would provide a range of patient authentication mechanisms customizable to meet patient needs and monitoring situations.

### SUMMARY OF THE INVENTION

A system and method includes passive and active authentication of patient data received or accepted from a source under remote patient management. Active authentication requires a patient to undertake a physical action, such as providing biometric, token, or code entry identifiers, which can provide identification credentials for comparison to authentication data prior to forwarding. Passive authentication utilizes credentialing indicia generally provided as an implantable device, such as an implantable medical device, implantable sensor, or implantable identification tag, to authenticate the physical proximity of a patient as the source of the patient data.

One embodiment provides a system and method for authenticating remotely collected external sensor measures. Physiological measures are collected from a source situated remotely from a repository for accumulating the physiological measures. The source of the physiological measures is identified by comparison to authentication data that uniquely identifies a specific patient. The physiological measures are forwarded to the repository upon authenticating the patient data as originating from the specific patient.

Still other embodiments of the present invention will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a functional block diagram showing, by way of example, an automated patient management environment.

FIG. 2 is a process flow diagram showing a method for providing authentication of remotely collected external sensor measures, in accordance with one embodiment.

FIG. 3 is a block diagram showing, by way of example, patient identification through passive authentication.

FIG. 4 is a block diagram showing, by way of example, patient identification through active authentication.

FIG. 5 is a functional block diagram showing patient identification with an external sensor.

FIG. 6 is a flow diagram showing patient identification with an external sensor.

FIG. 7 is a functional block diagram showing patient identification with a patient management device.

FIG. 8 is a flow diagram showing patient identification with a patient management device.

FIGS. 9, 11, and 13 are functional block diagrams showing patient identification with an implantable medical device.

FIGS. 10, 12, and 14 are flow diagrams showing patient identification with an implantable medical device.

FIGS. 15 and 16 are functional block diagrams showing patient identification for multiple patients, in accordance with one embodiment.

### DETAILED DESCRIPTION

Automated Patient Management Environment

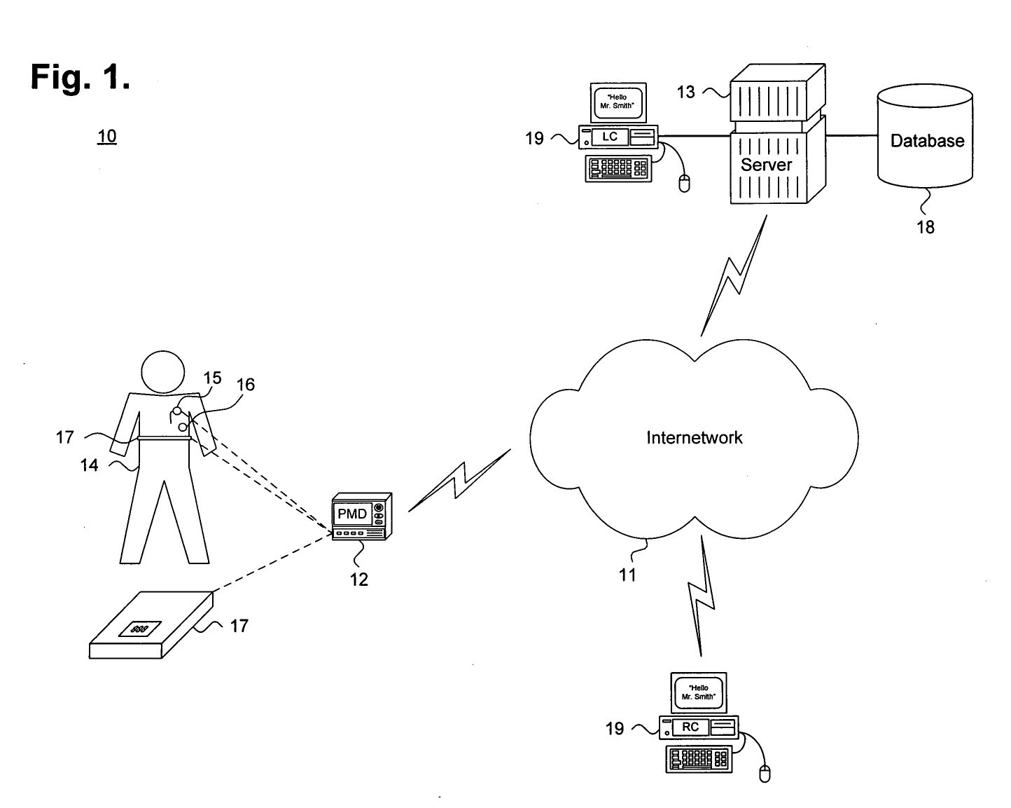

Automated patient management encompasses a range of activities, including remote patient management and automatic diagnosis of patient health, such as described in commonly-assigned U.S. Patent application Pub. No. US2004/0103001, published May 27, 2004, pending, the disclosure of which is incorporated by reference. Such activities can be performed proximal to a patient, such as in the patient's home or office, centrally through a centralized server, such from a hospital, clinic or physician's office, or through a remote workstation, such as a secure wireless mobile computing device. FIG. 1 is a functional block diagram showing, by way of example, an automated patient management environment 10. In one embodiment, a patient 14 is proximal to one or more patient monitoring or communications devices, such as a patient management device 12, which are interconnected remotely to a centralized server 13 over an internetwork 11, such as the Internet, or through a public telephone exchange (not shown), such as a conventional or mobile telephone network. Other patient monitoring or communications devices are possible. In addition, the functionality provided by the centralized server 13 could also be provided by local or decentralized servers, or by workstations, personal computers, or other computational systems accessible via the intemetwork 11 or other form of network. The internetwork 11 can provide both conventional wired and wireless interconnectivity. In one embodiment, the internetwork 11 is based on the Transmission Control Protocol/Internet Protocol (TCP/IP) network communication specification, although other types or combination of networking implementations are possible. Similarly, other network topologies and arrangements are possible.

Each patient management device 12 is uniquely assigned to a patient under treatment 14 to provide a localized and network-accessible interface to one or more medical devices 15-17, either through direct means, such as wired connectivity, or through indirect means, such as selective radio frequency or wireless telemetry based on, for example, “strong” Bluetooth or IEEE 802.11 wireless fidelity “WiFi” and “WiMax” interfacing standards. Other configurations and combinations of patient data source interfacing are possible. Medical therapy devices include implantable medical devices (IMDs) 15, such as pacemakers, implantable cardiac defibrillators (ICDs), drug pumps, and neuro-stimulators, as well as external medical devices (not shown). Medical sensors include implantable sensors 16, such as implantable heart and respiratory monitors and implantable diagnostic multi-sensor non-therapeutic devices, and external sensors 17, such as Holter monitors, weight scales, and blood pressure cuffs. Other types of medical therapy, medical sensing, and measuring devices, both implantable and external, are possible.

Patient data includes physiological measures, which can be quantitative or qualitative, parametric data regarding the status and operational characteristics of the patient data source itself, and environmental parameters, such as the temperature or time of day. In a further embodiment, patient data can also include psychological, drug dosing, medical therapy, and insurance-related information, as well as other types and forms of information, such as digital imagery or sound and patient-provided or -uploaded information. The medical devices 15-17 collect and forward the patient data either as a primary or supplemental function. The medical devices 15-17 include, by way of example, implantable and external medical therapy devices that deliver or provide therapy to the patient 14, implantable and external medical sensors that sense physiological data in relation to the patient 14, and measurement devices that measure environmental parameters and other data occurring independent of the patient 14. Other types of patient data are possible. Each medical device 15-17 can generate one or more types of patient data and can incorporate one or more components for delivering therapy, sensing physiological data, measuring environmental parameters, or a combination of functionality.

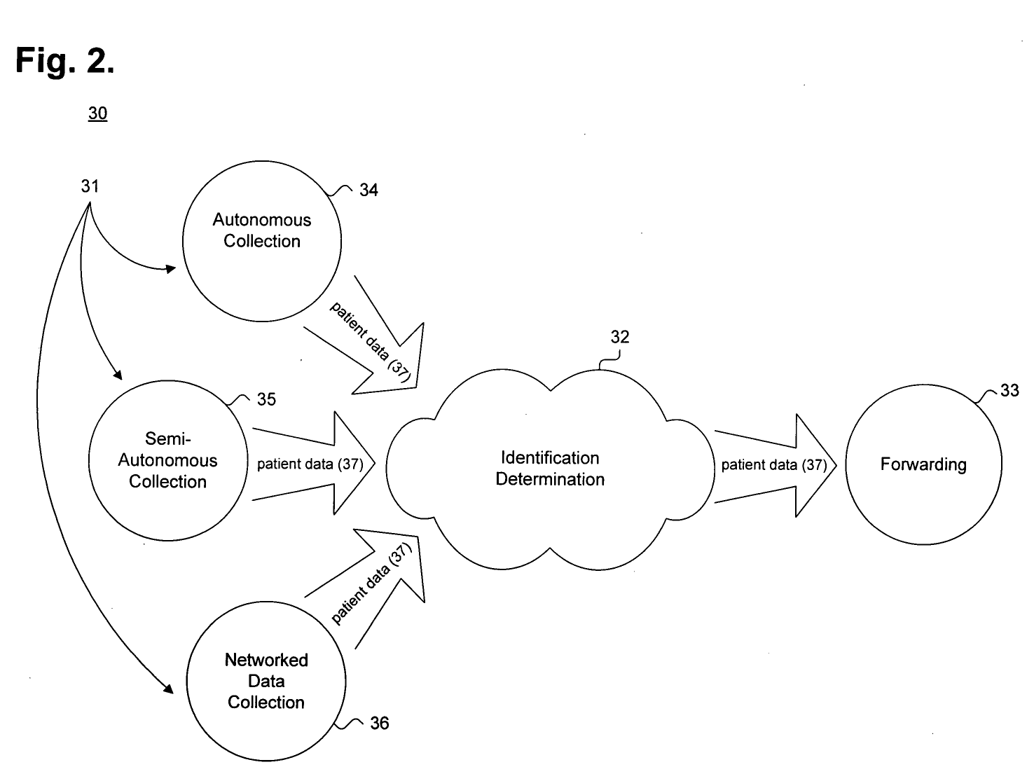

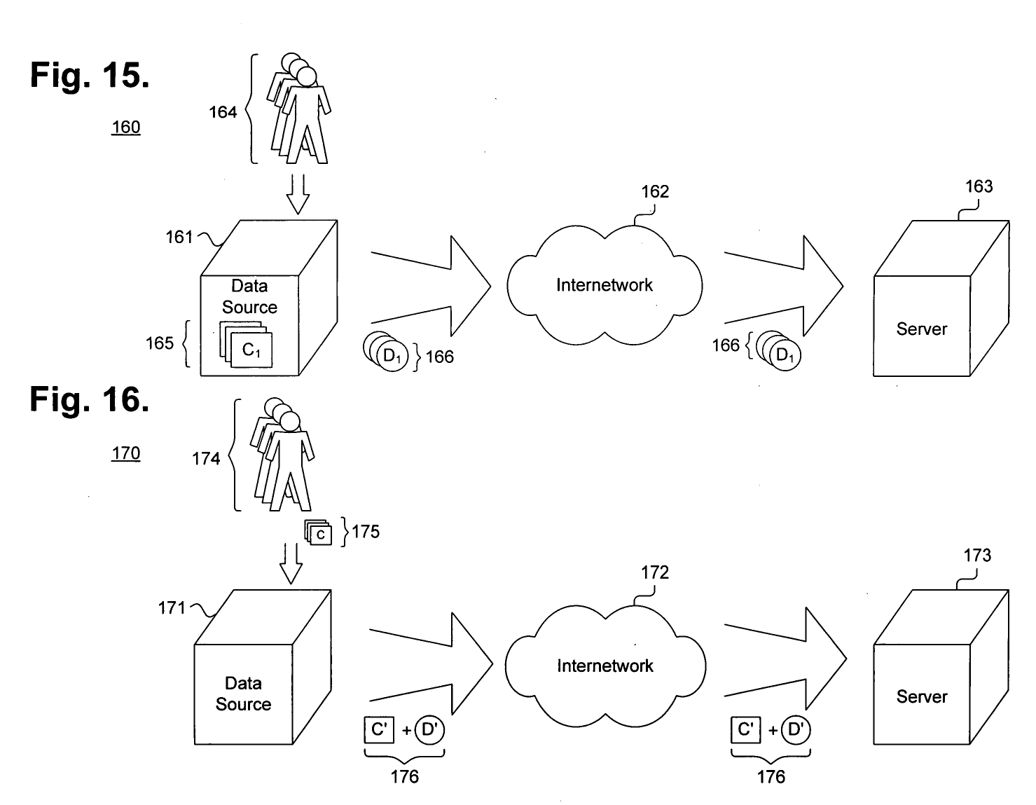

Patient data received from IMDs 15 and implantable sensors 16 is known to have originated from a particular patient 14, as implantable devices are uniquely identified by serial number or other identifying data. Accordingly, any patient data originating from an implantable device can only be from the patient 14 in which the device was implanted. Patient data received from external sensors 17, however, is not uniquely tied to a particular patient 14 and could instead originate from another person, such as a spouse or family member, or random source, such as a pet that accidentally triggers a sensor reading. To ensure the integrity of patient data, the identification of the source from which the patient data was collected is confirmed against authentication data that uniquely identifies a specific patient 14 prior to being forwarded to the centralized server 13 or other patient data repository. In one embodiment, a patient data source is associated with a specific patient in a one-to-one mapping that ensures authentication prior to receipt of the patent data at the centralized server 13, as further described below beginning with reference to FIG. 2. Briefly, patient data is received or collected and the forwarding of the patient data to the centralized server 13 or, in a further embodiment, the patient management device 12, is deferred until the identity of the source is locally authenticated through passive or active means. In a further embodiment, a single patient data source can be associated with multiple patients in a one-to-many mapping, such as further described below with reference to FIGS. 15 and 16.

In a further embodiment, data values can be directly entered by a patient 14. For example, answers to health questions could be input into a personal computer with user interfacing means, such as a keyboard and display or microphone and speaker. Such patient-provided data values could also be collected as patient information. In one embodiment, the medical devices 15-17 collect the quantitative physiological measures on a substantially continuous or scheduled basis and also record the occurrence of events, such as therapy or irregular readings. In a further embodiment, the patient management device 12, a personal computer, or similar device record or communicate qualitative quality of life (QOL) measures that reflect the subjective impression of physical well-being perceived by the patient 14 at a particular time. Other types of patient data collection, periodicity and storage are possible.

In a further embodiment, the collected patient data can also be accessed and analyzed by one or more clients 19, either locally-configured or remotely-interconnected over the internetwork 11. The clients 19 can be used, for example, by clinicians to securely access stored patient data assembled in a database 18 and to select and prioritize patients for health care provisioning, such as respectively described in commonly-assigned U.S. patent application, Ser. No. 11/121,593, filed May 3, 2005, pending, and U.S. patent application, Ser. No. 11/121,594, filed May 3, 2005, pending, the disclosures of which are incorporated by reference. Although described herein with reference to physicians or clinicians, the entire discussion applies equally to organizations, including hospitals, clinics, and laboratories, and other individuals or interests, such as researchers, scientists, universities, and governmental agencies, seeking access to the patient data.

In a further embodiment, patient data is safeguarded against unauthorized disclosure to third parties, including during collection, assembly, evaluation, transmission, and storage, to protect patient privacy and comply with recently enacted medical information privacy laws, such as the Health Insurance Portability and Accountability Act (HIPAA) and the European Privacy Directive. At a minimum, patient health information that identifies a particular individual with health- and medical-related information is treated as protectable, although other types of sensitive information in addition to or in lieu of specific patient health information could also be protectable.

Preferably, the server 13 is a computing platform configured as a uni-, multi- or distributed processing system, and the clients 19 are general-purpose computing workstations, such as a personal desktop or notebook computer. In addition, the patient management device 12, server 13 and clients 19 are programmable computing devices that respectively execute software programs and include components conventionally found in computing device, such as, for example, a central processing unit (CPU), memory, network interface, persistent storage, and various components for interconnecting these components.

Method Overview

Patient data includes any data that originates from a patient 14 under remote management and can include physiological measures, parametric data, and environmental parameters. The patient data can either be measured or generated directly by an external sensor 17 or can be submitted as already-measured values to a patient management device 12, either directly, such as through a user interface, or indirectly, via, for instance, an external sensor 17 or other device interfaced to the patient management device 12. FIG. 2 is a process flow diagram showing a method 30 for providing authentication of remotely collected external sensor measures, in accordance with one embodiment. External sensor measures include patient data that have been collected by a source other than an IMD 15 or implantable sensor 16, such as an external sensor 17.

By way of example, the collection 31 of patient data 37 can be performed autonomously 34, semi-autonomously 35, and through networked data collection 36. Autonomous patient data collection 34 is performed by an external sensor 17 independently from other devices and includes authentication of the source of the patient data 37, which is forwarded as a complete packet of information. Semi-autonomous data patient collection 35 is performed by an external sensor 17 in conjunction with another device, typically the patient management device 12, which uses the external sensor 17 as a measurement source and records the measurement as patient data 37. Networked data collection 36 is performed by a patient management device 12 or equivalent device, such as a Web-based personal computer, which receives the patient data 37 through a user interface, such as in response to queries presented to the patient 14. Other forms of patient data collection 31 are possible.

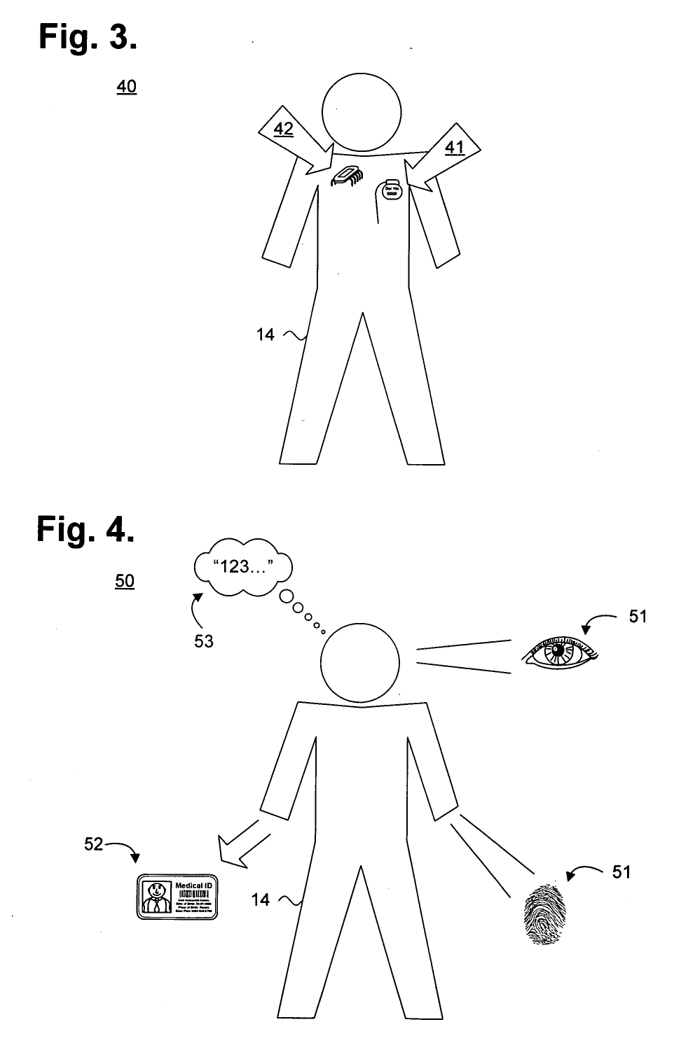

The delivery of the patient data 37 to the centralized server 13 and, in a further embodiment, a patient management device 12, is deferred pending the determination 32 of the identification of the source from which the patient data 37 was obtained. In one embodiment, identification determination 32 is performed passively by relying upon detectible indicia implanted physically into the patient 14, as further described below with reference to FIG. 3. In a further embodiment, identification determination 32 is performed actively by requiring the patient 14 to submit credentialing information, as further described below with reference to FIG. 4.

Following successful determination of the source of the patient data 37 as being the patient 14, the patient data 37 can be forwarded 33 for accumulation at the centralized server 13 or other repository to facilitate remote patient management. In further embodiments, the patient data 37 is forwarded on an interim basis to the patient management device 12 or to an IMD 15 or implantable sensor 16 for transient staging, pending eventual forwarding to the centralized server 13. Other forms of patient identification authentication are possible, including incremental or intermediate authentication on a point-to-point basis through passive, active, or combined authentication performed by one or more devices.

Passive Authentication

Passive authentication relies upon the presence of detectable indicia implanted into the patient 14 to provide the necessary authentication data by which to confirm patient identity. FIG. 3 is a block diagram showing, by way of example, patient identification through passive authentication 40. By way of example, detectable indicia can include a serial number or other uniquely identifying data internally associated with an IMD or implantable sensor 41, and an implantable identification tag 42, such as a radio frequency identification tag or similar device, which contains uniquely identifying data that can be remotely read. The identifying data is remotely accessed when the patient 14 is within sufficient proximity to ensure that the measurement originated with the patient 14 and not from another source.

The identifying data is compared against stored authentication data that uniquely identifies a specific patient 14. Passive authentication 40 requires the least amount of effort by the patient 14 and relies upon the system 10 to perform authentication transparently to the patient 14. However, the patient 14 must be willing to receive an implantable device, which contains the uniquely identifying data. Other forms of passive authentication are possible.

Active Authentication

Active authentication requires the patient 14 to undertake a physical action to provide credentialing information by which to confirm patient identity. FIG. 4 is a block diagram showing, by way of example, patient identification through active authentication 50. By way of example, active authentication 50 can utilize biometric identifiers 51, token identifiers 52, and code entry identifiers 53. Biometric identifiers 51 use a physical property of the patient 14, such as retina or iris pattern, fingerprint, voice pattern, personal identification number, or identification token, to uniquely identify the patient 14. However, biometric identifiers 51 may not be suitable for all patients 14, such as the infirm, elderly, or physically challenged. In a further embodiment, a token identifier 52, such as an identification card containing credentialing information, must be presented by the patient 14 prior to the system 10 accepting patient data for forwarding. Token identifiers 52, though, are susceptible to compromise, should the physical token be used by another person. In a still further embodiment, code entry identifier 53 assigns a personal identification number (PIN) or similar code to uniquely identify the patient 14. Code entry identifiers 53 are also susceptible to compromise, but can remain secure, as long as the patient 14 keeps the code identifier confidential. Other forms of active authentication are possible.

Patient Identification with an External Sensor

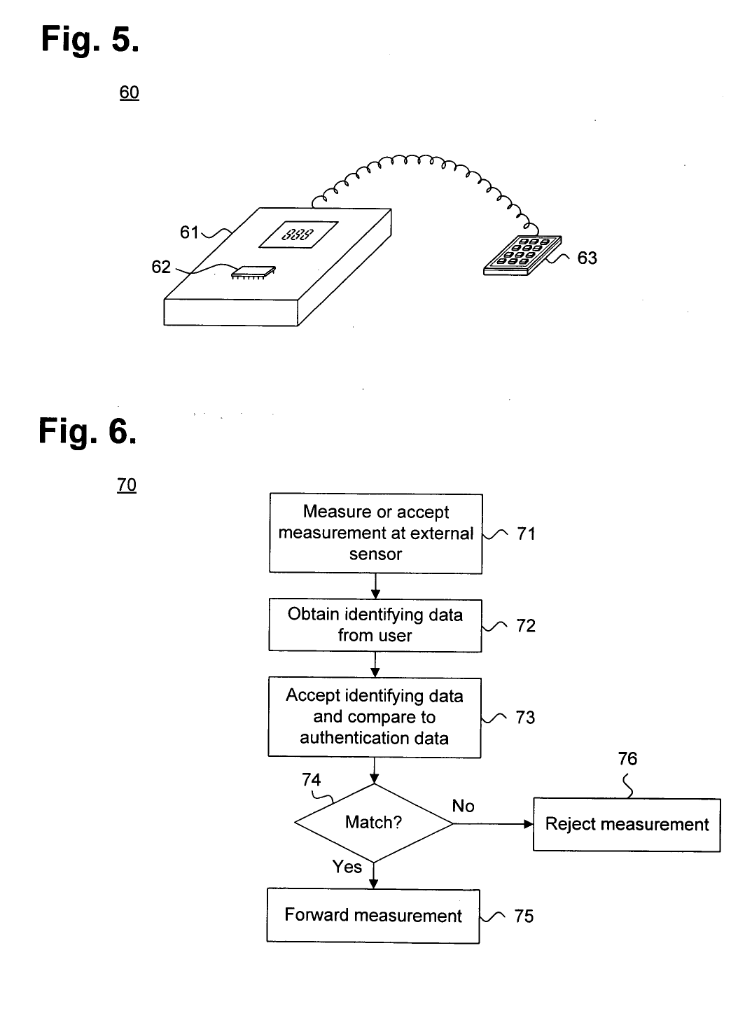

Autonomous patient data collection 34 (shown in FIG. 2) requires an external sensor to incorporate the capability of authenticating a patient. FIG. 5 is a functional block diagram showing patient identification 60 with an external sensor 61. The capability to authenticate a patient 14 is provided by supplementing the external sensor 61 with an input device 63, which can perform one or more forms of active patient identification, such as receiving a retina or iris pattern, fingerprint, voice pattern, personal identification number, or identification token. The external sensor 61 also stores authentication data 62 that is maintained in a form suitable for automated comparison to the results of the input device 63. Suitable input devices include a retinal or iris scanner, fingerprint scanner, voice input device, keypad, barcode scanner, or magnetic card reader. Other forms of input devices for active patient identification and for storing correspondingly suitable authentication data are possible.

Autonomous patient data collection 34 is performed by the external sensor 61 independent from the centralized server, patient management device, and other devices. The external sensor 61 defers forwarding the collected patient data to the patient management device 12 or, in a further embodiment, the centralized server 13, pending confirmation of patient identity. FIG. 6 is a flow diagram showing patient identification 70 with an external sensor 61. Initially, a measurement is measured or accepted by the external sensor 61 (block 71). The measurement can be displayed, but will not be forwarded from the external sensor 61, pending authentication of the identity of the source from which the measurement was collected. Identifying data is solicited and obtained from the user (block 72), such as by prompt or displayed message. Identifying data provided by the user is accepted and compared to the authentication data 62 (block 73). If the identifying data matches the authentication data 62 (block 74), the measurement is forwarded (block 75) to the patient management device 12 or, in a further embodiment, the centralized server 13. Otherwise, the measurement is rejected (block 76).

Patient Identification with a Patient Management Device

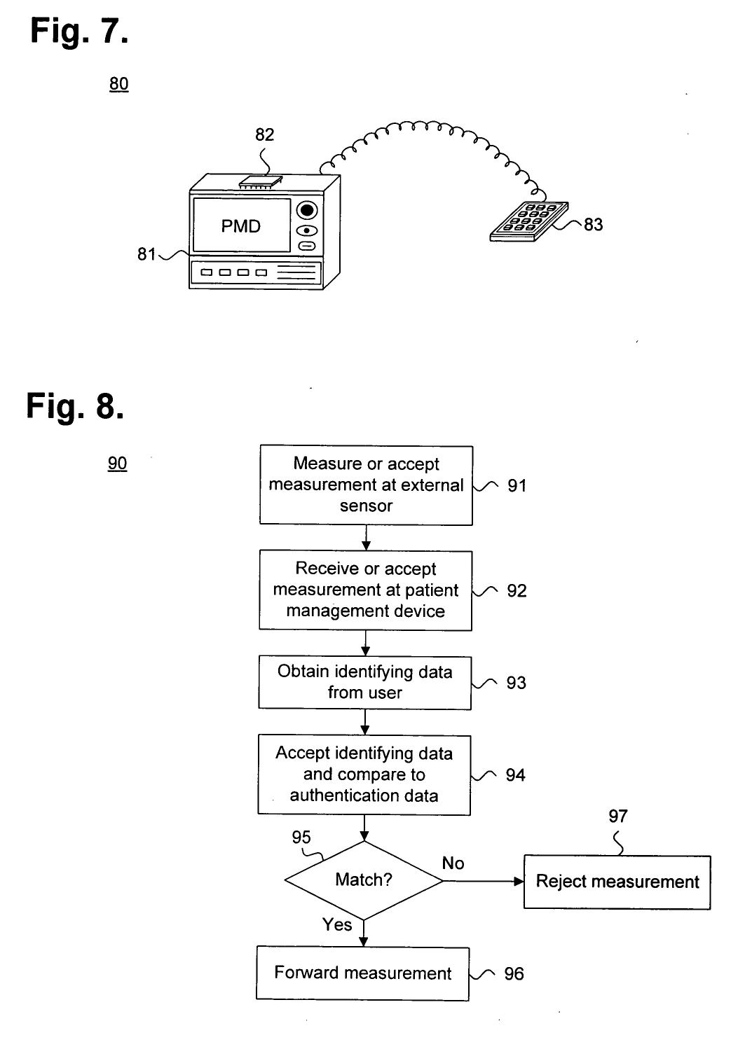

Patient management devices must also include the capability to confirm patient identification when performing semi-autonomous patient data collection 35 or networked data collection 36. FIG. 7 is a functional block diagram showing patient identification 80 with a patient management device 81. The capability to authenticate a patient 14 is provided by supplementing the patient management device 81 with an input device 83, which can perform one or more forms of active patient identification, such as receiving a retina or iris pattern, fingerprint, voice pattern, personal identification number, or identification token. The patient management device 81 also stores authentication data 82 that is maintained in a form suitable for automated comparison to the results of the input device 83. Suitable input devices include a retinal or iris scanner, fingerprint scanner, voice input device, keypad, barcode scanner, or magnetic card reader. Other forms of input devices for active patient identification and for storing correspondingly suitable authentication data are possible.

Similar to the autonomous patient data collection 34 performed by an external sensor 61, each patient management device 81 defers forwarding the collected patient data to the centralized server 13 pending confirmation of patient identity. FIG. 8 is a flow diagram showing patient identification 90 with a patient management device. Initially, a measurement is measured or accepted by an external sensor 17 (block 91) and is received or accepted at the patient management device 81 (block 92). The measurement can be displayed, but the measurement will not be forwarded from the patient management device 81, pending authentication of the identity of a source from which the measurement was collected. Identifying data is solicited and obtained from the user (block 93), such as by prompt or displayed message. Identifying data provided by the user is accepted and compared to the authentication data 82 (block 94). If the identifying data matches the authentication data 82 (block 95), the measurement is forwarded (block 96) to the centralized server 13. Otherwise, the measurement is rejected (block 97).

Patient Identification with an Implantable Medical Device

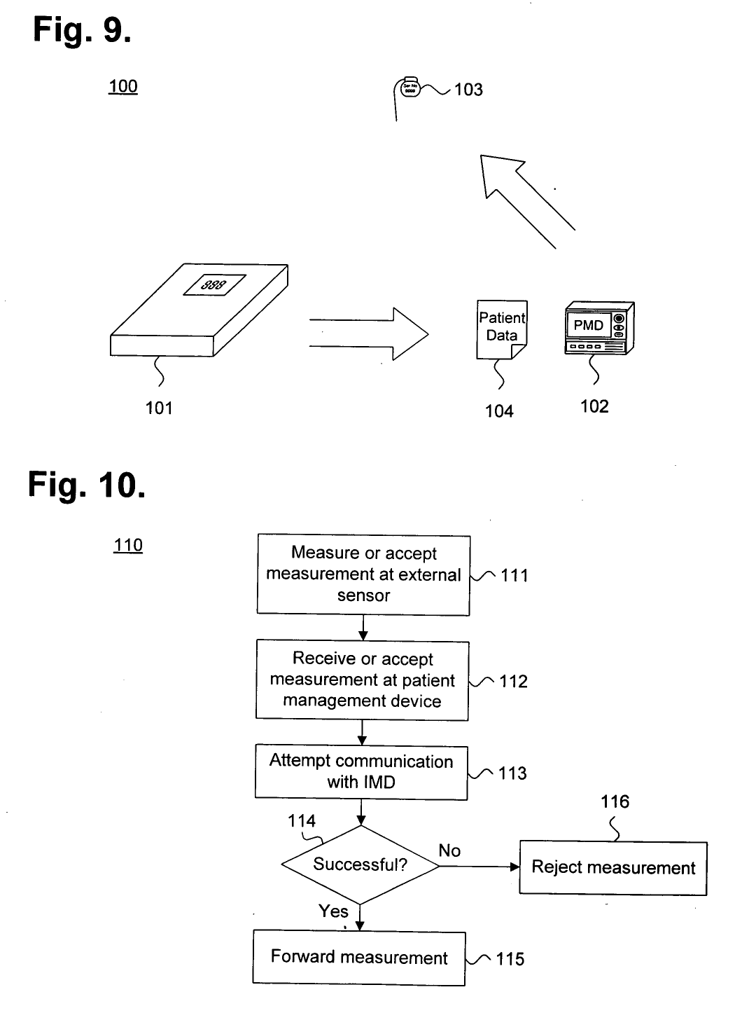

Passive authentication requires detectable indicia generally available through a device implanted in the patient 14, such as an IMD, implantable sensor, or implantable identification tag. FIGS. 9, 11, and 13 are functional block diagrams showing patient identification 100, 120, 140 with an implantable medical device 103, 123, 143. A separate input device is not required, as the implantable medical device itself serves as the device by which patient identity is confirmed. FIGS. 10, 12, and 14 are flow diagrams showing patient identification 110, 130, 150 with an implantable medical device 103, 123, 143. The implantable device containing the detectable indicia is referred to generally as an implantable medical device, but also includes implantable sensors, implantable identification tags, and other forms of implantable devices that can be uniquely associated with a patient 14 through remote detection.

Prior to being forwarded to the centralized server 13, the patient data can be transiently staged at either an external sensor, patient management device, or implantable medical device. Transiently staging patient data at a patient management device enables the patient data to be forwarded to the centralized server immediately upon authentication, but consumes storage on the patient management device if the authentication fails and the patient data must ultimately be discarded as spurious. Referring to FIG. 9, a patient management device 102 that is in receipt of patient data 104 received or accepted from an external sensor 101 confirms the presence of an implantable medical device 103. The patient management device 102 utilizes near field telemetry, such as induction, or far field telemetry, such as radio frequency communication, to attempt to communicate with the implantable medical device 103. A failure of communication implies that the implantable medical device 103 and, therefore, the patient 14 are not present and the patient data 104 is discarded as spurious.

Referring next to FIG. 10, initially, a measurement is measured or accepted by the external sensor 101 (block 111) and is received or accepted by the patient management device 102 (block 112). Upon receiving the measurement, the patient management device 102 attempts to communicate with the implantable medical device 103 (block 113). If the communication attempt is successful (block 114), the measurement is forwarded by the patient management device 102 as patient data 104 to the centralized server 13 (block 115). Otherwise, the measurement is rejected as spurious (block 116).

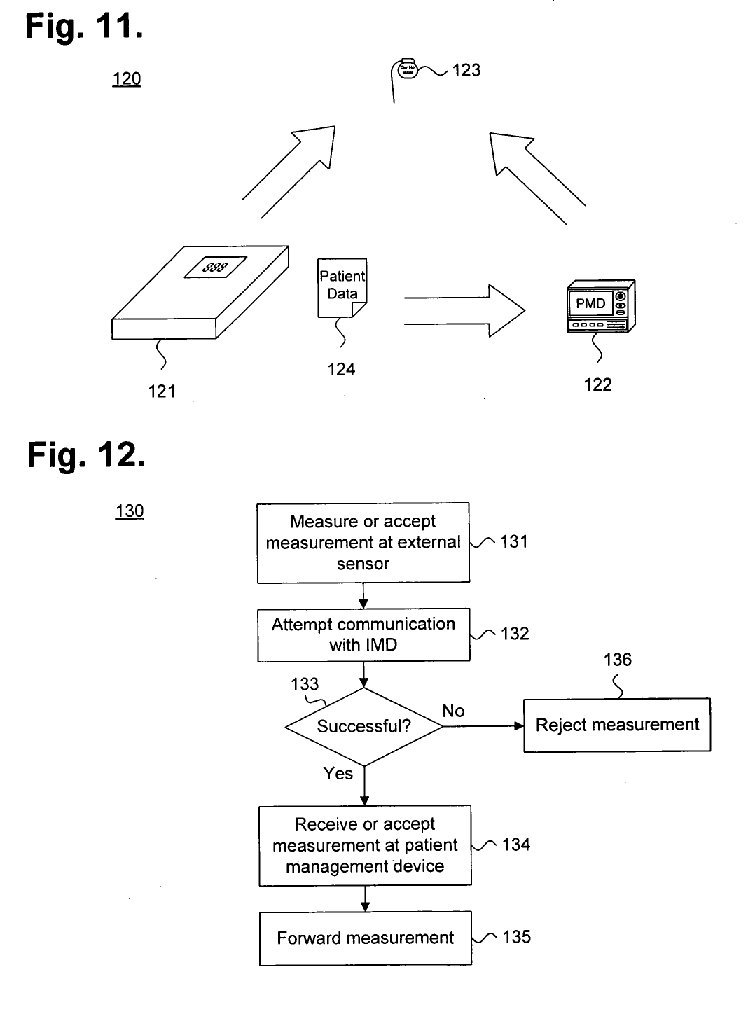

Transiently staging the patient data on an external sensor avoids consuming storage on a patient management device if authentication fails, but can incur a delay in forwarding the patient data to the centralized server while the patient data is forwarded from the external sensor to the patient management device. Referring next to FIG. 11, an external sensor 121 that has measured or accepted patient data 124 confirms the presence of an implantable medical device 123. The external sensor 121 utilizes near field telemetry, such as induction, or far field telemetry, such as radio frequency communication, to attempt to communicate with the implantable medical device 123. A failure of communication implies that the implantable medical device 123 and, therefore, the patient 14 are not present and the patient data 124 is discarded as spurious. Discarded patient data is never actually received by the patient management device 122.

Referring next to FIG. 12, initially, a measurement is measured or accepted by the external sensor 121 (block 131). The external sensor 121 attempts to communicate with the implantable medical device 123 (block 132). If the communication attempt is successful (block 133), the measurement is received or accepted by the patient management device 122 (block 134) and is forwarded to the centralized server 13 (block 135). Otherwise, the measurement is rejected as spurious (block 136).

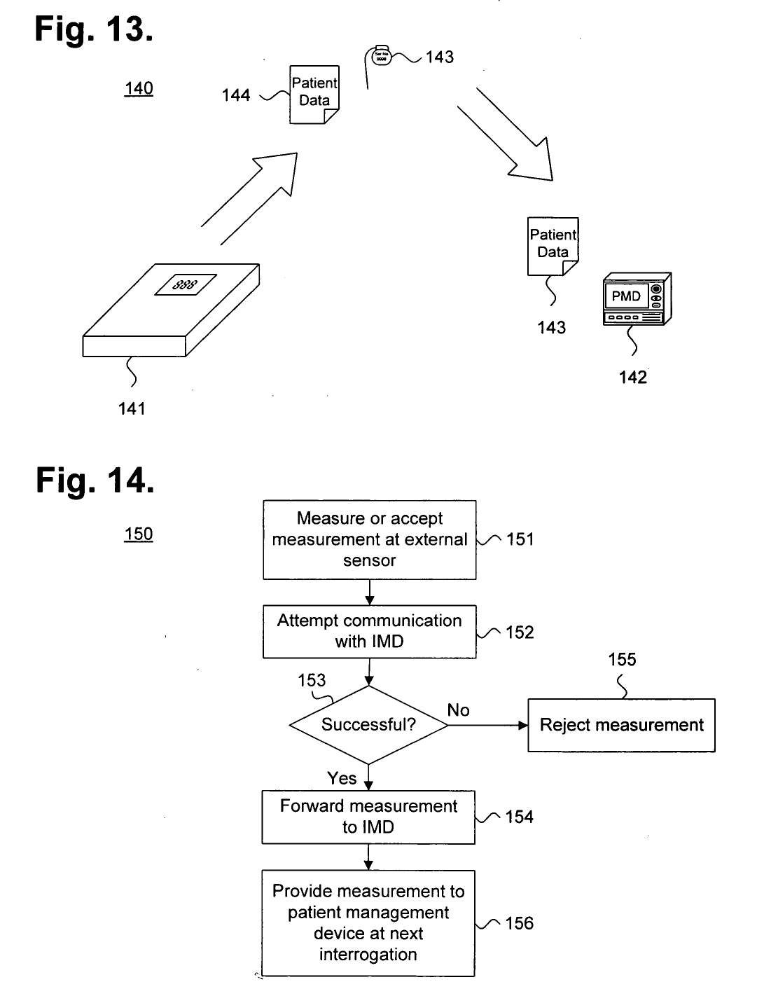

Transiently staging the patient data on an implantable medical device avoids involving a patient management device in authentication, but is expensive in terms of the resources consumed, as the implantable medical device must expend processing, storage, and power budget resources to temporarily hold the patient data pending forwarding to the patient management device. The implantable medical device must have sufficient resources to temporarily hold the patient data pending upload to the patient medical device. Referring next to FIG. 13, an external sensor 141 sends patient data 144 to an implantable medical device 143 that is implanted in the patient 14. To guard against patient data being uploaded to the implantable medical device 143 that originated from a source other than the patient 14, the external sensor 141 and implantable medical device 143 must be in close physical proximity so as to ensure that the patient data source is the patient with the implantable medical device 143. The requirement for close physical proximity implicitly provides patient identification authentication and, accordingly, the patient data 143 can be forwarded to the patient management device 142 at the next interrogation for eventual forwarding to the centralized server 13.

Referring next to FIG. 14, the external sensor 141, patient management device 142, and implantable medical device 143 each participate, but only the implantable medical device 143 directly interfaces to both the external sensor 141 and patient management device 142. Initially, a measurement is measured or accepted by the external sensor 141 (block 151), which then attempts to communicate with the implantable medical device 143 (block 152). In one embodiment, the external sensor 141 uses near field telemetry, such as induction, to ensure close physical proximity of the patient 14. In a further embodiment, the external sensor 141 uses far field telemetry, such as radio frequency communication, that is set to a short transmission range to ensure close physical proximity of the patient 14. If the communication attempt is successful (block 153), the measurement is forwarded to the implantable medical device 143 (block 154) and is eventually provided to the patient management device 142 at the next data interrogation (block 156) for eventual forwarding to the centralized server 13. Otherwise, the measurement is implicitly rejected as spurious through non-delivery to the implantable medical device 143 (block 155).

Patient Identification for Multiple Patients

In one embodiment, a single patient data source can be associated with a specific patient in a one-to-one mapping, which provides local authentication. In a further embodiment, a single patient data source can be associated with multiple patients in a one-to-many mapping. FIGS. 15 and 16 are functional block diagrams showing patient identification 160, 170 for multiple patients, in accordance with one embodiment. One-to-many mappings can be used, for example, to enable multiple patients to share a single external medical sensor 17 or, where appropriate, external medical devices, or to provide data from an implantable or external medical sensor or device, subject to proper credentialing. Referring first to FIG. 15, patient identification 160 through local authentication can be provided by maintaining multiple sets of patient credentials 165 at a patient data source 161. Each patient credentials set 165 is associated with a specific patient 164 and the patient data source 161 accepts readings of physiological measures only from those users authorized through the maintained patient credentials sets 165. Authenticated patient data 166 is forwarded to a centralized server 163 through an internetwork 162, which can include one or more intermediate patient management devices (not shown).

Referring next to FIG. 16, patient identification 170 through remote authentication can be provided by accepting a set of credentials 175 associated with a particular patient 174 at a patient data source 171. The patient data source 171 or, in a further embodiment, a patient management device (not shown), will reject patient data collected or read from users that fail to provide authenticating patient credentials sets. Otherwise, physiological data and the patient credentials set 176 are forwarded to a centralized server 173 over an intemetwork 172, which can include one or more intermediate patient management devices (not shown), for authentication by a centralized server 173. The centralized server 173 will authenticate patient data collected or read from authorized users and will reject patient data collected or read from unauthorized users. Other one-to-many, as well as many-to-many, mappings are possible.

While the invention has been particularly shown and described as referenced to the embodiments thereof, those skilled in the art will understand that the foregoing and other changes in form and detail may be made therein without departing from the spirit and scope of the invention.

What is claimed is:

1. A system for providing authentication of remotely collected external sensor measures, comprising:
a collection module to collect physiological measures from a source situated remotely from a repository for accumulating such collected physiological measures;
an identification module to determine an identification of the source from which the physiological measures were collected against authentication data that uniquely identifies a specific patient; and
a staging module to forward the physiological measures to the repository upon authenticating the patient identification as originating from the specific patient.

2. A system according to claim 1, wherein the collection module comprises at least one of a discrete external sensor, an external sensor operatively coupled to a patient management device, and a patient management device passively receiving the physiological measures.

3. A system according to claim 2, wherein the external sensor comprises at least one of a weight scale, blood pressure cuff, glucometer, thermometer, and spirometer.

4. A system according to claim 1, wherein the patient identification is passively determined through use of at least one of an implantable medical device and radio frequency identification tag, which each comprise authentication data.

5. A system according to claim 1, wherein the patient identification is actively determined through use of at least one of a biometric identifier, token identifier, and code entry identifier, which each provide credentials for comparison to the authentication data.

6. A system according to claim 1, wherein the staging module comprise at least one of an external sensor, patient management device, and implantable medical device.

7. A system according to claim 1, further comprising:
an external sensor on which to implement the authentication data;
a store to hold the physiological measures on the external sensor until the patient identification is confirmed, wherein the physiological measures are provided to a patient management device upon confirmation.

8. A system according to claim 1, further comprising:
a patient management device on which to implement the authentication data;
an external sensor to provide the physiological measures to the patient management device; and
a store to hold the physiological measures on the patient management device until the patient identification is confirmed.

9. A system according to claim 1, further comprising:
an external sensor to provide the physiological measures to a patient management device;
a communications module to confirm proximity of an implantable medical device to the patient management device; and
a store to hold the physiological measures on the patient management device until the implantable medical device proximity is confirmed.

10. A system according to claim 1, further comprising:
a communications module to confirm communication between an implantable medical device and an external sensor, wherein the physiological measures are provided to a patient management device upon confirmation.

11. A system according to claim 1, further comprising:
a communications module to confirm communication between an implantable medical device and an external sensor, wherein the physiological measures are provided to the implantable medical device upon confirmation.

12. A system according to claim 1, further comprising:
patient data included with the forwarded physiological measures comprising at least one of psychological, drug dosing, medical therapy, insurance-related, digital imagery or sound, and patient-provided or -uploaded information.

13. A method for providing authentication of remotely collected external sensor measures, comprising:
collecting physiological measures from a source situated remotely from a repository for accumulating such collected physiological measures;
determining an identification of the source from which the physiological measures were collected against authentication data that uniquely identifies a specific patient; and
forwarding the physiological measures to the repository upon authenticating the patient identification as originating from the specific patient.

14. A method according to claim 13, further comprising:
collecting the physiological measures using at least one of a discrete external sensor, an external sensor operatively coupled to a patient management device, and a patient management device passively receiving the physiological measures.

15. A method according to claim 14, wherein the external sensor comprises at least one of a weight scale, blood pressure cuff, glucometer, thermometer, and spirometer.

16. A method according to claim 13, further comprising:
passively determining the patient identification through use of at least one of an implantable medical device and radio frequency identification tag, which each comprise authentication data.

17. A method according to claim 13, further comprising:
actively determining the patient identification through use of at least one of a biometric identifier, token identifier, and code entry identifier, which each provide credentials for comparison to the authentication data.

18. A method according to claim 13, further comprising:
forwarding the physiological measures from at least one of an external sensor, patient management device, and implantable medical device.

19. A method according to claim 13, further comprising:
implementing the authentication data on an external sensor;
holding the physiological measures on the external sensor until the patient identification is confirmed; and
providing the physiological measures to a patient management device upon confirmation.

20. A method according to claim 13, further comprising:
implementing the authentication data on a patient management device;
providing the physiological measures to the patient management device from an external sensor; and
holding the physiological measures on the patient management device until the patient identification is confirmed.

21. A method according to claim 13, further comprising:
providing the physiological measures to a patient management device from an external sensor;
confirming proximity of an implantable medical device to the patient management device; and
holding the physiological measures on the patient management device until the implantable medical device proximity is confirmed.

22. A method according to claim 13, further comprising:
confirming communication between an implantable medical device and an external sensor; and
providing the physiological measures to a patient management device upon confirmation.

23. A method according to claim 13, further comprising:
confirming communication between an implantable medical device and an external sensor; and
providing the physiological measures to the implantable medical device upon confirmation.

24. A method according to claim 13, further comprising:
including patient data with the forwarded physiological measures comprising at least one of psychological, drug dosing, medical therapy, insurance-related, digital imagery or sound, and patient-provided or -uploaded information.

25. A computer-readable storage medium holding code for performing the method according to claim 13.

26. An apparatus for providing authentication of remotely collected external sensor measures, comprising:
means for collecting physiological measures from a source situated remotely from a repository for accumulating such collected physiological measures;
means for determining an identification of the source from which the physiological measures were collected against authentication data that uniquely identifies a specific patient; and
means for forwarding the physiological measures to the repository upon authenticating the patient identification as originating from the specific patient.
